(19) United States
(12) Patent Application Publication
(10) Pub. No.: US 2024/0221910 A1
(43) Pub. Date: Jul. 4, 2024

(54) SYSTEM AND METHOD FOR EARLY DETECTION OF A PSYCHOTHERAPEUTIC TREATMENT RESPONSE

(71) Applicant: TALIAZ LTD., Tel Aviv (IL)

(72) Inventor: Dekel TALIAZ, Modi'in (IL)

(21) Appl. No.: 18/287,509

(22) PCT Filed: Apr. 14, 2022
(86) PCT No.: PCT/IL2022/050393

§ 371 Date: Oct. 19, 2023

Related U.S. Application Data
(60) Provisional application No. 63/178,039, filed on Apr. 22, 2021.

(51) Int. Cl. G16H 20/70; G16H 10/60

(52) U.S. Cl. CPC G16H 20/70; G16H 10/60

(57) ABSTRACT

There is provided herein a method for early detection of a psychotherapeutic treatment response for a subject in need thereof, the method including obtaining data associated with a level of severity of a mental health of the subject four weeks or less after initiation of the psychotherapeutic treatment, calculating a post-initiation score associated with the subject's mental health based on the obtained data, and classifying the subject as responsive or non-responsive to the specified psychotherapeutic treatment based, at least in part, on a comparison of the post-initiation score to a score associated with the mental health of comparable subjects that have received the specified psychotherapeutic treatment.

### TECHNICAL FIELD

The present disclosure relates generally to systems and methods for early detection of a subject's response to a psychotherapeutic treatment.

### BACKGROUND

Mood disorders are among the most prevalent forms of mental illness. Severe forms of mental illness affect 2%-5% of the US population and up to 20% of the population suffers from milder forms of the illness. The economic costs to society and personal costs to individuals and families are enormous.

Anti-depressants are a primary method for treatment of depression. Anti-depressant drugs are known to influence the functioning of certain monoamine neurotransmitters, primarily serotonin, norepinephrine, and dopamine. Older medications, such as tricyclic anti-depressants (TCAs) and monoamine oxidase inhibitors (MAOIs), affect the activity of all these neurotransmitters simultaneously. However, these medications can be difficult to tolerate due to side effects or, in the case of MAOIs, dietary and medication restrictions. Newer medications, such as selective serotonin reuptake inhibitors (SSRIs) norepinephrine reuptake inhibitors (NRIs), Serotonin-norepinephrine reuptake inhibitors (SNRIs), Norepinephrine-Dopamine Reuptake Inhibitors (NDRIs) and Serotonin-Norepinephrine-Dopamine Reuptake Inhibitors (SNDRIs), Mirtazapine, Nefazodone, Trazodone, and Vortioxetine also have side-effects, though fewer. Prescription of anti-depressant medication is often inexact and their efficacy is assessed empirically. Depression, as well as other prevalent psychiatric disorders, is characterized by a high degree of variability in patient response to the drugs administered, even among individuals with the same diagnosis. In fact, only roughly 35% of patients demonstrate complete remission following first prescribed treatment. Furthermore, some patients respond, but with serious adverse side effects.

Current methods for selecting a suitable depression treatment are basically trial-and-error. Patients will often have to be treated with several kinds of medicine, before finding the most suitable drug. This is obviously a problem, which is further augmented by the fact that four to six weeks of chronic treatment are required to evaluate the anti-depressant phenotype, the efficacy, and effectiveness, of the treatment and whether an adverse event is registered. It is therefore not surprising that patients tend to cease taking their medications against medical advice.

However, there remains an unmet medical need for a method capable of early detection of the efficacy and adverse effects of an anti-depressant treatment in a patient suffering from a psychiatric disorder such as depression. This is important in order to shorten the time required to achieve an optimal treatment regime with minimal adverse side effects.

### SUMMARY

According to some embodiments, the present disclosure provides a method for early detection of a responsiveness of a subject being treated using one or more psychotherapeutic treatments at an early stage of the treatment. According to some embodiments, the method includes obtaining data, from the treated subject, associated with a mental health of the subject, within four weeks (e.g. four weeks or less, three weeks or less or two weeks or less) of initiation of the psychotherapeutic treatment, and using the data to classify the subject as being responsive or non-responsive to the psychotherapeutic treatment during future weeks of the psychotherapeutic treatment (such as weeks 4 to 12 or 6 to 12 of the treatment).

According to some embodiments, the method may include generating a score associated with the mental health of the subject and comparing the score with scores associated with the mental health of comparable subjects that have received the psychotherapeutic treatment, thereby enabling a rapid assessment of the psychotherapeutic treatment efficiency for the treated subject. The method may therefore prevent long durations of treatment for a subject in order to assess his or her responsiveness, thereby enabling the subject to receive another treatment in a case in which the subject may be identified as a non-responsive to the specified treatment, allowing a quicker improvement in the mental health of the subject overall. Since many of the psychotherapeutic treatments have different side effects, the method therefor enables the improvement of the compliance of the psychotherapeutic treatments to the subject by increasing the likelihood of the treated subject to receive a suitable treatment to which the subject will be responsive, thereby preventing the subject from giving up treatment before finding the right treatment match to which the subject will be responsive.

Advantageously, the method enables taking into account different trends in the treatment process of comparable subjects, such as variance in the responsiveness of the comparable subjects over time, to identify responsiveness of the treated subject within two to four weeks of initiating the psychotherapeutic treatment. The method thereby enables early detection of the responsiveness of the subject to a psychotherapeutic treatment without having to finish a full course of the treatment. Advantageously, for a subject that may be classified as unresponsive to a first treatment within two to four weeks of initiation thereof, the subject will not need to continue the treatment and may be offered a different psychotherapeutic treatment, thereby preventing a loss of time, motivation, or even unwanted side effects of the first treatment.

According to some embodiments, the method may include scoring the mental health of the subject based on any one or more of data inputted by the subject, such as answers to one or more questionnaires, passively collected data, or behavioral data, such as data associated with smartphone usage, sleeping patterns, GPS tracking, sleep characteristics, and level of activity, demographic data such as employment status, residence, private health care insurance, age, and marital status, clinical data such as the medical file/chart of the subject, medical history of the subject, and medical conditions of the subject.

According to some embodiments, the method may include classifying the subject as responsive and/or non-responsive to a psychotherapeutic treatment using a mathematical model, such as an exponential model, based on the obtained score associated with the mental health of the subject. Advantageously, classifying the subject based on the model results in higher accuracy of early detection, as well as sensitivity of the early detection, than commonly used classic detection techniques.

According to some embodiments, the method may include comparing the score of the subject to scores of comparable subjects, wherein the comparable subjects may have similarities, and the similarities may include any one or more of similar and/or comparable psychotherapeutic treatments, passively collected data, demographic data, and/or clinical data, thereby enabling higher detection accuracy and sensitivity for the early detection of responsiveness of a subject to a specified psychotherapeutic treatment. Advantageously, comparing the score of the subject to scores of comparable subjects enables higher detection accuracy and sensitivity in classification of the treated subject as a responder or non-responder to the psychotherapeutic treatment, within four or even two weeks from initiation of the psychotherapeutic treatment.

According to some embodiments of the present invention there is provided a method for early detection of a psychotherapeutic treatment response for a subject in need thereof, the method including: obtaining data associated with a level of severity of a mental health of the subject four weeks or less after initiation of the psychotherapeutic treatment, calculating a post-initiation score associated with the subject's mental health based on the obtained data, and classifying the subject as responsive or non-responsive to the specified psychotherapeutic treatment based, at least in part, on a comparison of the post-initiation score to a score associated with the mental health of comparable subjects that have received the specified psychotherapeutic treatment.

According to some embodiments, the predetermined time includes two weeks or less after initiation of psychotherapeutic treatment. According to some embodiments, the obtained data includes subjective and/or objective data associated with the level of severity of a mental health of the subject. According to some embodiments, the subjective obtained data includes data inputted by the subject. According to some embodiments, the objective obtained data includes passive/behavioral data collected from the subject. According to some embodiments, the passive data includes sleep characteristics, level of activity, and smartphone usage. According to some embodiments, the data may include demographic data of the subject. According to some embodiments, the data further includes clinical data of the subject. According to some embodiments, the classifying may be based on a pre-known ratio of responsive to non-responsive subjects to the specified psychotherapeutic treatment.

According to some embodiments, the classifying further includes taking into account the initial level of severity of the subject in relation to the initial level of severity associated with the plurality of scores associated with comparable subjects that have received the specified psychotherapeutic treatment. According to some embodiments, the comparable subjects include subjects having any one or more of a similar and/or comparable level of severity, symptoms, duration of symptoms prior to initiation of treatment, age, medical history, sex, and behavioral characteristics. According to some embodiments, the post-initiation score and the score associated with the mental health of comparable subjects that have received the specified psychotherapeutic treatment, are based on different types of data.

According to some embodiments, the method may include obtaining data associated with a level of severity of a mental health of the subject at another time within four or less weeks of initiation of the specified psychotherapeutic treatment, and calculating an initial treatment score associated with the subject's mental health based on the obtained data. According to some embodiments, the method may include obtaining data associated with a level of severity of a mental health of the subject prior to the initiation of the specified psychotherapeutic treatment, and calculating an enrollment score associated with the subject's mental health based on the obtained data. According to some embodiments, the post-initiation score, the initial treatment score, and/or the enrollment score are based on different types of data.

According to some embodiments, the post-initiation score, the initial treatment score, the enrollment score, and/or the score associated with the mental health of comparable subjects that have received the specified psychotherapeutic treatment, are based on different types of data. According to some embodiments, the method may include generating a non-linear model based, at least in part, on the post-initiation score and the initial treatment score and/or the enrollment score. According to some embodiments, the non-linear model is an exponential model.

According to some embodiments, the method may include deriving a parameter from the non-linear model. According to some embodiments, the parameter includes at least one of a slope, area under curve, plateau, maximal slope, minimal slope, average slope, median slope, standard deviation, correlation coefficient and variance. According to some embodiments, the parameter is associated with a degree of change in a behavior of the subject. According to some embodiments, the method may include providing a weight to the parameter and/or to the post-initiation score and/or the initial treatment score and/or the enrollment score based on a general change in behavior of a population of individuals to which the subject belongs, the population of individuals including individuals who are not diagnosed with a mental disease.

According to some embodiments, the method may include classifying the subject as responsive or non-responsive to the specified psychotherapeutic treatment based, at least in part, on a comparison of the parameter to a parameter associated with the mental health of comparable subjects that have received the specified psychotherapeutic treatment. According to some embodiments, the comparable subjects include subjects that had a previous response to other psychotherapeutic treatment(s) similar and/or comparable to a previous response of the treated subject, to a similar and/or comparable psychotherapeutic treatment(s), prior to and/or during receiving the specified psychotherapeutic treatment and/or a psychotherapeutic treatment comparable with the psychotherapeutic treatment.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1 shows a schematic illustration of steps of an exemplary method for early detection of a psychotherapeutic treatment response for a subject, in accordance with some embodiments of the present invention; and

FIG. 2 shows a schematic illustration of a chart showing exemplary correlations within and/or between behavioral parameters and other obtained data of the subject, in accordance with some embodiments of the present invention.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

As used herein, the term “anti-depressant treatment” refers to drugs used in the treatment of patients suffering from depression. Antidepressants are known to influence the functioning of certain monoamine neurotransmitters, primarily serotonin, norepinephrine, and dopamine. Older medications, such as tricyclic anti-depressants (TCAs) and monoamine oxidase inhibitors (MAOIs), affect unspecific combinations of these neurotransmitters simultaneously. Newer medications comprise selective serotonin reuptake inhibitors (SSRIs), norepinephrine-selective reuptake inhibitors (NRIs), Norepinephrine-Dopamine Reuptake Inhibitors (NDRIs), Serotonin-Norepinephrine-Dopamine Reuptake Inhibitor (SNDRI), Mirtazapine, Nefazodone, Trazodone, and Vortioxetine.

As used herein, the term “psychiatric disorders” refers to any psychiatric disorders including, but not limited to, depression, attention deficit disorder, schizophrenia, bipolar disorder, anxiety disorders, substance use disorder, eating disorders such as anorexia nervosa and bulimia nervosa, phobias, dissociative disorders, insomnia, and borderline personality disorder.

As used herein, the terms “depression,” “depressive disorder,” and “mood disorder” interchangeably refer to a DSM-IV and/or DSM-V definition of depression. It is to be understood that depression comprises different subtypes such as Atypical depression (AD), Melancholic depression, Psychotic major depression (PMD), Catatonic depression, Postpartum depression (PPD), Seasonal affective disorder (SAD), Dysthymia, Depressive Disorder Not Otherwise Specified (DD-NOS), Recurrent brief depression (RBD), Major depressive disorder and Minor depressive disorder; which all fall under the scope of the invention.

Atypical depression (AD) is characterized by mood reactivity (paradoxical anhedonia) and positivity, significant weight gain or increased appetite (“comfort eating”), excessive sleep or somnolence (hypersomnia), a sensation of heaviness in limbs known as leaden paralysis, and significant social impairment as a consequence of hypersensitivity to perceived interpersonal rejection.

Melancholic depression is characterized by a loss of pleasure (anhedonia) in most or all activities, a failure of reactivity to pleasurable stimuli, a quality of depressed mood more pronounced than that of grief or loss, a worsening of symptoms in the morning hours, early-morning waking, psychomotor retardation, excessive weight loss, or excessive guilt.

Psychotic major depression (PMD), or simply psychotic depression, is the term for a major depressive episode, in particular of melancholic nature, wherein the patient experiences psychotic symptoms such as delusions or, less commonly, hallucinations.

Catatonic depression is a rare and severe form of major depression involving disturbances of motor behavior and other symptoms. Here, the person is mute and almost stuporous, and either is immobile or exhibits purposeless or even bizarre movements.

Postpartum depression (PPD) refers to the intense, sustained and sometimes disabling depression experienced by women after giving birth. Seasonal affective disorder (SAD), also known as “winter depression” or “winter blues”, refers to depressive episodes coming on in the autumn or winter, and resolving in spring.

Dysthymia is a chronic, different mood disturbance where a person reports a low mood almost daily over a span of at least two years. The symptoms are not as severe as those for major depression.

Depressive Disorder Not Otherwise Specified (DD-NOS) refers to disorders that are impairing but do not fit any of the officially specified diagnoses.

Recurrent brief depression (RBD) is distinguished from major depressive disorder primarily by differences in duration. People with RBD have depressive episodes about once per month, with individual episodes lasting less than two weeks and typically less than 2-3 days.

As used herein the term “clinical data” may refer to any non-genetic parameter influencing the subject's response to a psychotherapeutic treatment. According to some embodiments, the term “clinical feature” may include physiological features (e.g., pain) and psychological features (e.g., anxiety), as further described herein below.

As used herein the term “demographic data” may refer to any non-genetic parameter associated with an environment of the subject. According to some embodiments, the term “demographic feature” may include sociological features (e.g., marital status) and economical features (e.g., employment status), as well as behavioral characteristics of the subject, as further described herein below.

As used herein the term “behavioral data” or “passive data” may refer to any habit, routine, or custom of the subject. According to some embodiments, the term “behavioral data” or “passive data” may include cell phone usage, internet usage habits, sleep habits, activity levels, and one or more analyses derived from data extracted from cellphone and/or internet usage habits.

It is to be understood that “responsive” to psychotherapeutic treatment as used herein does not necessarily mean that the subject will benefit from the psychotherapeutic treatment, but rather that the subject is, in a statistical sense, more likely to belong to the class of patients that will benefit from the psychotherapeutic treatment.

The term “classification algorithm” as used herein refers to methods that implement a model (classifier) for predicting a discrete category or class membership (target label), to which the data belong. The term “classification algorithm” may include, but is not limited to, machine learning algorithms.

The term “machine learning algorithm” as used herein refers to a construction of a method (algorithm) that can learn from and make predictions on data.

The term “score” as used herein refers to the total score that each subject receives from the method, which may be quantified in order to detect a response outcome of the subject.

As used herein, the term “non-responsive” refers to a subject who has experienced one or more scores associated with being unresponsive to one or more psychotherapeutic treatments within a period of time such as two weeks or four weeks within initiation of the psychotherapeutic treatment, or such as 6 to 12 weeks after initiation of the psychotherapeutic treatment.

The term “exponential modeling” as used herein refers to a model that fits the data exponentially, this will suit cases where the data change by a fixed (or close to fixed) percentage.

As used herein, the term “classic response” with regards to a subject's response to a treatment refers to an improvement of 50% or more in the subject's depression symptoms score. Additionally or alternatively, the efficacy may be determined according to a curve taking into consideration both the depression symptoms score as well as time of treatment. The efficacy of the treatment is determined quantitatively by a score of one or more rating scales, such as the Hamilton Rating Scale for Depression (HAM-D), QUICK INVENTORY OF DEPRESSIVE SYMPTOMATOLOGY (QIDS), Patient Health Questionnaire-9 (PHQ-9), Patient Health Questionnaire-8 (PHQ-8), Beck's Depression Inventory (BDI), Emotional State Questionnaire, international classification of diseases (ICD), Diagnostic and Statistical Manual of Mental Disorders (DSM, such as, e.g., DSM-III and DSM-5), or Global Clinical Impression Scale. The HAM-D scale contains items that assess somatic symptoms, insomnia, working capacity and interest, mood, guilt, psychomotor retardation, agitation, anxiety, and insight. As used herein a 50% decrease in the HAM-D, QIDS or the BDI score is considered an efficient treatment response. The degree of adverse side effects of anti-depressant treatment is determined quantitatively by the Udvalg Kliniske Undersøgelser (UKU) Side Effect Rating Scale, the Frequency and Intensity of Side Effects Rating (FISER) or the Global Rating of Side Effects Burden (GRSEB) scales. Each possibility is a separate embodiment of the invention.

As used herein, the term “psychotherapeutic treatment” refers to treatments such as antidepressant treatments, cognitive behavioral therapy (CBT), and electroconvulsive therapy (ECT).

According to some embodiments, the subject in need of the psychiatric or psychotherapeutic treatment may suffer from a psychiatric disorder selected from the group consisting of depression, attention deficit disorder, schizophrenia, bipolar disorder, anxiety disorders, substance use disorder, eating disorders such as anorexia nervosa and bulimia nervosa, phobias, dissociative disorders, insomnia, and borderline personality disorder or any combination thereof. According to some embodiments, the subject is suffering from depression and the psychiatric drug is an anti-depressant. According to some embodiments, the antidepressant is selected from the group consisting of: citalopram, paroxetine, sertraline, zimelidine, escitalopram, indalpine, dapoxetine, fluvoxamine, fluoxetine, talopram, talsupram, reboxetine, viloxazine, atomoxetine, bupropion, desoxypipradrol, edivoxetine, amedalin, desvenlafaxine, milnacipram, daledalin, venlafaxine, duloxetine, tandamine, lortalamine, levomilnacipran, difemetorex, dexmethylphenidate, maprotiline, mirtazapine, nefazodone, trazodone, and vortioxetine and any combination thereof. According to some embodiments, the anti-depressant is citalopram.

According to some embodiments, the subject in need of the psychotherapeutic treatment may suffer from any one or more of psychiatric disorders, depression, Atypical depression, Melancholic depression, Psychotic major depression, Catatonic depression, Postpartum depression (PPD), Seasonal affective disorder (SAD), Dysthymia, Depressive Disorder Not Otherwise Specified (DD-NOS), and Recurrent brief depression (RBD).

The term “classic response detection” refers to a commonly used technique for detection of responsiveness of a subject to a treatment, in which subjects are scored based on a questionnaire, and a “classic responder” refers to an improvement of 50% or more in the subject's depression score.

The term “sensitivity” or “true positive rate” refer to a measure of the proportion of positives that are correctly identified, such as the proportion of subjects that are responsive to a treatment and are classified as responders to the treatment.

The term “specificity” or “true negative rate” refer to a measure of the proportion of negatives that are correctly identified, such as the proportion of subjects that are non-responsive to a treatment and are classified as non-responders to the treatment.

The term “balanced accuracy” refers to an average of the sensitivity and the specificity.

As used herein the term “detection”, “detecting”, or “detect” a response refers to concluding a subject's future response type (e.g., responder or non-responder) to a treatment, after the subject has begun treatment.

As used herein the term “early detection” of a response of a subject refers to concluding a subject's future response type (e.g., responder or non-responder) to a treatment, wherein the early detection is made within four and/or within two weeks after intake (or time in which the subject has initiated treatment).

As used herein the term “predicting”, such as predicting a response of the treated subject to a second psychotherapeutic treatment and predicting a second psychotherapeutic treatment for which the treated subject will have a higher responsiveness level, refers to forecasting a response and/or second psychotherapeutic treatment prior to the subject's initiation of the second treatment, and/or based on data that was obtained prior to the subject's initiation of the second treatment.

According to some embodiments, there is provided a method for early detection of a response to psychotherapeutic treatment in a subject in need thereof. Advantageously, the methods disclosed herein enable detecting the patient's response to treatment within four weeks, two weeks, or one week, after initiating the treatment. According to some embodiments, the method may indicate the response of a subject to one or more psychotherapeutic treatment based on data obtained from the subject, for example, by using inputted data from the subject and/or passive data collected from the subject. That is, the methods enable detecting the patient's psychotherapeutic treatment response based on a combination of obtained data, wherein the data may be obtained, for example, through a patient' answers to a questionnaire, a tracking of the patient's electronic device usage, sleeping patterns, and the like.

According to some embodiments, the method may include scoring the obtained data using a model configured to unify different types of obtained data into a score, thereby enabling the comparison of different subject's potential responses to psychotherapeutic treatments. According to some embodiments, the method may include scoring the obtained data, thereby generating a score associated with the subject's mental health. According to some embodiments, the method may include comparing the score of a subject to any one of more of a model and/or database associated with a same treatment, a same patient background, and the like. According to some embodiments, and as described in greater detail elsewhere herein, the model and/or database may include one or more scores associated with the mental health of comparable subjects that have received the specified psychotherapeutic treatment. According to some embodiments, and as described in greater detail elsewhere herein, the method may include selecting a subset of comparable subjects. According to some embodiments, the model and/or database may include one or more scores associated with the mental health of the selected subset of comparable subjects.

According to some embodiments, the method may include classifying the subject as a responder or non-responder to one or more psychotherapeutic treatment. According to some embodiments, the method may include detecting the subject's future response to one or more psychotherapeutic treatment. According to some embodiments, the method may include determining the treatment outcome of the subject for one or more psychotherapeutic treatment.

According to some embodiments, digital-behavioral biomarkers, or in other words, behavioral data, can not only assist in the diagnosis of disorders, but can also enable new characterization of subtypes of such disorders.

According to some embodiments, applying an algorithm on the database of subjects enables sub-typing different disorders into sub-categories. According to some embodiments, the method may include analyzing the obtained data to classify a subject's disorder into a sub-category. According to some embodiments, the method may include classifying the diagnosed disorder of the subject to one or more sub-categories associated with the diagnosed disorder. According to some embodiments, the classification into sub-categories may be based, at least in part, on any one or more of the symptoms of the subject and severity of the symptoms of the subject. According to some embodiments, the classification into sub-categories may be based, at least in part, on the collected data. According to some embodiments, the classification into sub-categories may be based on obtained and/or collected data, such as, for example, passively collected data from the subject.

Advantageously, classifying the subject's disorder into one or more sub-categories may increase the efficacy of treatment of the subject.

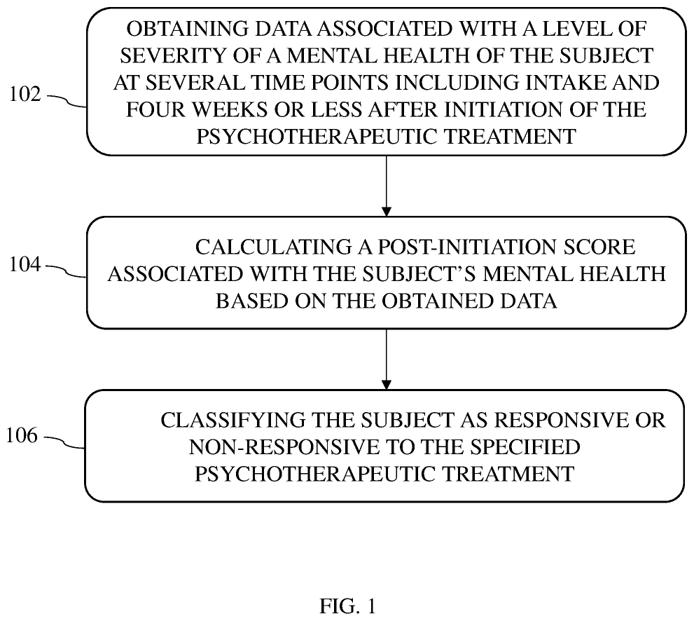

According to some embodiments, the method may include selecting a sub-section of data of the obtained data based, at least in part, on the classified sub-category of the disorder of the subject. According to some embodiments, the method may include calculating the post-initiation score based, at least in part, on the selected sub-section of data of the obtained data. According to some embodiments, the method may include calculating the enrollment score based, at least in part, on the selected sub-section of data of the obtained data. According to some embodiments, the method may include calculating the initial treatment score based, at least in part, on the selected sub-section of data of the obtained data. Reference is made to FIG. 1, which shows a schematic illustration of steps of an exemplary method for early detection of a psychotherapeutic treatment response for a subject, in accordance with some embodiments of the present invention.

According to some embodiments, at step 102, the method may include obtaining data associated with a level of severity of a mental health of the subject at several time points including intake and four weeks or less after initiation of the psychotherapeutic treatment. According to some embodiments, at step 104, the method may include calculating a post-initiation score associated with the subject's mental health based on the obtained data. According to some embodiments, at step 106, the method may include classifying the subject as responsive or non-responsive to the specified psychotherapeutic treatment.

According to some embodiments, there is provided a system for detecting a psychotherapeutic treatment response for a subject. According to some embodiments, the system may include at least one hardware processor. According to some embodiments, the system may include a non-transitory computer-readable storage medium having stored thereon program code. According to some embodiments, the program code may be executable by the at least one hardware processor to obtain data associated with a level of severity of a mental health of the subject four weeks or less after initiation of the psychotherapeutic treatment. According to some embodiments, the program code may be executable by the at least one hardware processor to calculate a post-initiation score associated with the subject's mental health based on the obtained data. According to some embodiments, the program code may be executable by the at least one hardware processor to classify the subject as responsive or non-responsive to the specified psychotherapeutic treatment.

According to some embodiments, the system may include a memory module configured to store data associated with subjects that have received and/or are currently receiving one or more psychotherapeutic treatments. According to some embodiments, the memory module may include a database of subjects that have received one or more psychotherapeutic treatments. According to some embodiments, the database may include one or more of the clinical data, demographic data, and/or behavioral data of the subjects that have received one or more psychotherapeutic treatments.

According to some embodiments, the program code may be executable by the at least one hardware processor to execute instructions associated with detecting (e.g., early-detection) and/or classifying the psychotherapeutic treatment response for a subject. According to some embodiments, the program code may include instructions configured to execute the method for early detection of a psychotherapeutic treatment response for a subject, such as method 100. According to some embodiments, and as described in greater detail elsewhere herein, the program code may include an algorithm, such as a classifying algorithm, configured to classify a subject as responsive or non-responsive to a psychotherapeutic treatment based, at least in part, on data associated with the mental health of the treated subject.

According to some embodiments, at step 102, the method may include obtaining data associated with a level of severity of a mental health of the subject at several time points including intake (or in other words, prior to initiation of the treatment) and four weeks or less after initiation of the psychotherapeutic treatment. According to some embodiments, the method may include obtaining data associated with a level of severity of a mental health of the subject two weeks or less after initiation of the psychotherapeutic treatment. According to some embodiments, the method may include obtaining data associated with a level of severity of a mental health of the subject one week or less after initiation of the psychotherapeutic treatment.

According to some embodiments, the obtained data may include subjective and/or objective data associated with the level of severity of a mental health of the subject. According to some embodiments, the obtained data may be inputted by the subject. According to some embodiments, the method may include receiving input from the subject associated with the level of severity of a mental health of the subject. According to some embodiments, the obtained data may be passively collected. According to some embodiments, the method may include collecting data from the subject, such as, for example, data associated with smartphone usage, sleeping patterns, GPS tracking, and the like. According to some embodiments, the objective data may include passive and/or behavioral data collected from the subject. According to some embodiments, the passive data may include sleep characteristics, level of activity, and smartphone usage. According to some embodiments, the obtained data may include longitudinal data. According to some embodiments, the obtained data may include passively collected longitudinal behavioral data.

According to some embodiments, the one or more behavioral data may be derived from data associated with computer usage and/or cell phone usage of the subject. According to some embodiments, the behavioral data of the subject may be monitored via a computer and/or cell phone of the subject. According to some embodiments, the behavioral data of the subject may be analyzed using data received from one or more electronic devices used by the subject. For example, according to some embodiments, the behavioral data may be derived from data associated with an internet history of the subject. For example, according to some embodiments, the behavioral data may be derived from data associated with social media associated with the subject. According to some embodiments, the behavioral data may be derived from GPS tracking of the subject.

According to some embodiments, the obtained data may include demographic data of the subject. According to some embodiments, the demographic data may include any one or more of employment status, residence, private health care insurance, age, marital status, one or more behavioral characteristics of the subject, or any combination thereof. According to some embodiments, the obtained data may include clinical data of the subject. According to some embodiments, the clinical data may include any one or more of the medical file/chart of the subject, medical history of the subject, and medical conditions of the subject. For example, in some embodiments, the obtained data may include any one or more of diagnosis of diabetes, autoimmune disease, heart disease, and the like.

According to some embodiments, the method may include storing the obtained data within a database associated with the treated subject. According to some embodiments, the method may include storing the obtained data continuously or semi-continuously.

According to some embodiments, the method may include deriving the level of severity from the obtained data. According to some embodiments, the method may include deriving the level of severity from any one or more of behavioral data, passively collected data, demographic data, questionnaires (such as QIDS or Hamilton), or any combination thereof. Every possibility is a separate embodiment.

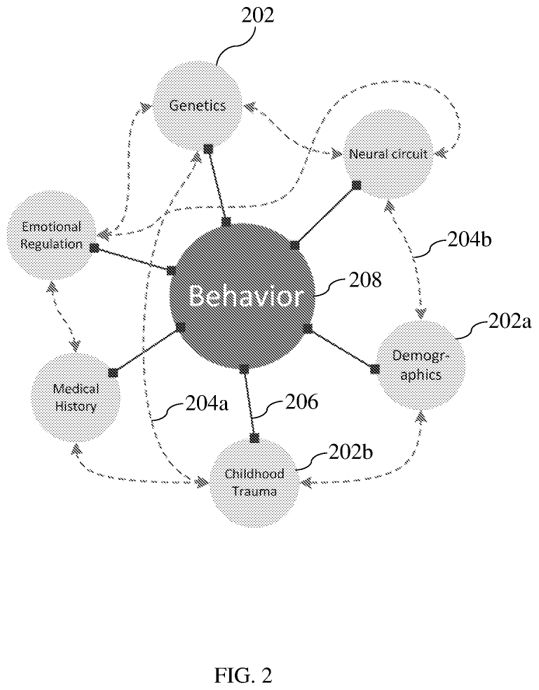

For example, according to some embodiments, the method may include deriving the level of severity by analyzing the patient's behavior by collecting behavioral data through different technological methods (e.g. smartphone apps, smart-watch, Electronic Health Record (EHR), and the like). Reference is now made to FIG. 2, which shows a schematic illustration of a chart showing exemplary correlations within and/or between behavioral parameters and other obtained data of the subject, in accordance with some embodiments of the present invention.

According to some embodiments, the behavioral data may include one or more behavioral parameters 208, such as, for example, sleep quantity and/or quality, activity levels (such as, for example, activity levels during the day and/or night), social interactions (e.g., daily social interactions), light exposure, and the like. According to some embodiments, the method may include analyzing the obtained data. According to some embodiments, the method may include cross-referencing (such as depicted by arrows 206) between behavioral parameters 208 of the behavioral data to other parameters associated with the obtained data (such as depicted within the circles 202). According to some embodiments, the other parameters associated with the obtained data may include demographic data, the subject's medical history, childhood trauma, genetics, emotional regulation, neural circuit, and the like, such as depicted within the circles 202 of FIG. 2. For example, the emotional regulation and/or emotional state of the subject may be derived using data of the subject's mobility and smartphones' normal usage, to predict an individual's mood state changes.

According to some embodiments, the method may include deriving one or more correlations within and/or between one or more behavioral parameters of the behavioral data to one or more other parameters associated with the obtained data. According to some embodiments, the method may include deriving one or more correlations and/or trends within an individual parameter (such as one or more behavioral parameter or one or more of the other parameters associated with the obtained data). Each possibility is a separate embodiment of the invention. As a non-limiting example, deriving one or more trend within an individual parameter may include identifying a trend (e.g., a decline in social interactions, an increase in sleep hours, and the like). According to another non limiting example, deriving the one or more correlation within an individual parameter may include identifying a pattern (e.g., the subject makes a call to the same person every day, the subject wakes up every morning at a certain time, the subject is regularly late to appointments, and the like). According to some embodiments, the method may include deriving one or more correlations between two or more of the other parameters associated with the obtained data (such as the correlations depicted by lines 204). According to some embodiments, the one or more correlations and/or trends may be a function of the treatment period (such as the number of weeks of treatment). As a non-limiting example, a correlation as a function of treatment period may include a decrease in sleep hours (that may be linear or non-linear) that continuous to decrease with continuation of the subject's treatment, and may also plateau after reaching a certain time after initiation of the treatment. According to another non limiting example, a correlation as a function of treatment period may include an increase in social activity (such as meeting people, phone calls, and/or texts) as the treatment progresses.

According to some embodiments, the method may include identifying one or more additional parameters and/or additional data that needs to be obtained. Each possibility is a separate embodiment of the invention.

As a non-limiting example, identifying one or more additional parameters and/or additional data that needs to be obtained may include identifying a need to collect data associated with identification of items/products that were ordered or bought by the subject. According to another non-limiting example, identifying one or more additional parameters and/or additional data that needs to be obtained may include identifying a need to collect data associated with types of food which the subject consumes.

Advantageously, deriving correlations within and/or between one or more behavioral parameters of the behavioral data to one or more other parameters associated with the obtained data enables administration of treatment that is specifically tailored to the subject, or in other words, enables personalized psychotherapeutic treatment for the subject.

Advantageously, the derived correlations may be used to increase the efficacy of one or more treatments of the subject by associating the one or more derived correlations with one or more sub-categories of disorders.

According to some embodiments, at step 104, the method may include calculating a post-initiation score associated with the subject's mental health based on the obtained data. According to some embodiments, the post-initiation score may be based on data collected over a period of an hour, 1-23 hours, a day, 1-5 days, and/or more than 1-5 days. According to some embodiments, the obtained data may include data associated with one or more of the subjective data, the objective data, the behavioral data, the demographic data, the clinical data, or any combination thereof. According to some embodiments, the method may include correlating the post-initiation score to the obtained data. According to some embodiments, the post-initiation score may include a rank, such as, for example, on a scale of 1 to 50. According to some embodiments, the post-initiation score may include a vector and/or an array. According to some embodiments, and as described in greater detail elsewhere herein, the post-initiation score may include a trend line and/or a line fitting calculation. According to some embodiments, the post-initiation score may have consistent units regardless of the data on which it is based. For example, according to some embodiments, a first post-initiation score of a first subject, wherein the first post-initiation score is based on demographic data of the first subject, may have a same unit(s), or in other words, be comparable to, a second post-initiation score of a second subject, wherein the second post-initiation score may be based on clinical data.

According to some embodiments, the method may include obtaining enrollment data (also referred to herein as intake data) associated with a level of severity of a mental health of the subject prior to the initiation of the specified psychotherapeutic treatment. According to some embodiments, the method may include collecting the enrollment data over a period of an hour, 1-23 hours, a day, 1-5 days and/or more than 1-5 days. According to some embodiments, the method may include calculating an enrollment score associated with the subject's mental health based on the obtained enrollment data associated with a level of severity of a mental health of the subject prior to the initiation of the specified psychotherapeutic treatment. According to some embodiments, the enrollment data may include subjective and/or objective data associated with the level of severity of a mental health of the subject. According to some embodiments, the enrollment data may be inputted by the subject. According to some embodiments, the method may include receiving input from the subject associated with the level of severity of a mental health of the subject. According to some embodiments, the enrollment data may be passively collected. According to some embodiments, the method may include collecting data from the subject, such as, for example, data associated with smartphone usage, sleeping patterns, GPS tracking, and the like. According to some embodiments, the objective data may include passive and/or behavioral data collected from the subject. According to some embodiments, the passive data may include sleep characteristics, level of activity, and smartphone usage.

According to some embodiments, the one or more behavioral data may be derived from data associated with computer usage and/or cell phone usage of the subject. According to some embodiments, the behavioral data of the subject may be monitored via a computer and/or cell phone of the subject. According to some embodiments, the behavioral data of the subject may be analyzed using data received from one or more electronic devices used by the subject. For example, according to some embodiments, the behavioral data may be derived from data associated with an internet history of the subject. For example, according to some embodiments, the behavioral data may be derived from data associated with social media associated with the subject. According to some embodiments, the behavioral data may be derived from GPS tracking of the subject.

According to some embodiments, the enrollment data may include demographic data of the subject. According to some embodiments, the demographic data may include any one or more of employment status, residence, private health care insurance, age, marital status, one or more behavioral characteristics of the subject, or any combination thereof. According to some embodiments, the enrollment data may include clinical data of the subject. According to some embodiments, the clinical data may include any one or more of the medical file/chart of the subject, medical history of the subject, and medical conditions of the subject. For example, in some embodiments, the enrollment data may include any one or more of diagnosis of diabetes, autoimmune disease, heart disease, and the like.

According to some embodiments, the method may include obtaining initial treatment data associated with a level of severity of a mental health of the subject at a second time within four weeks of initiation of the specified psychotherapeutic treatment. According to some embodiments, the initial treatment data may be obtained before or after the obtained data associated with the post-initiation score. According to some embodiments, the method may include calculating an initial treatment score associated with the subject's mental health based on the obtained initial treatment data of/at the second time within four weeks of initiation of the specified psychotherapeutic treatment. According to some embodiments, the post-initiation score, the initial treatment score, and/or the enrollment score are based on different types of data associated with the subject.

According to some embodiments, the initial treatment data may include subjective and/or objective data associated with the level of severity of a mental health of the subject. According to some embodiments, the initial treatment data may be inputted by the subject. According to some embodiments, the method may include receiving input from the subject associated with the level of severity of a mental health of the subject. According to some embodiments, the initial treatment data may be passively collected. According to some embodiments, the method may include collecting data from the subject, such as, for example, data associated with smartphone usage, sleeping patterns, GPS tracking, and the like. According to some embodiments, the objective data may include passive and/or behavioral data collected from the subject. According to some embodiments, the passive data may include sleep characteristics, level of activity, and smartphone usage.

According to some embodiments, the one or more behavioral data may be derived from data associated with computer usage and/or cell phone usage of the subject. According to some embodiments, the behavioral data of the subject may be monitored via a computer and/or cell phone of the subject. According to some embodiments, the behavioral data of the subject may be analyzed using data received from one or more electronic devices used by the subject. For example, according to some embodiments, the behavioral data may be derived from data associated with an internet history of the subject. For example, according to some embodiments, the behavioral data may be derived from data associated with social media associated with the subject. According to some embodiments, the behavioral data may be derived from GPS tracking of the subject.

According to some embodiments, the initial treatment data may include demographic data of the subject. According to some embodiments, the demographic data may include any one or more of employment status, residence, private health care insurance, age, marital status, one or more behavioral characteristics of the subject, or any combination thereof. According to some embodiments, the initial treatment data may include clinical data of the subject. According to some embodiments, the clinical data may include any one or more of the medical file/chart of the subject, medical history of the subject, and medical conditions of the subject. For example, in some embodiments, the initial treatment data may include any one or more of diagnosis of diabetes, autoimmune disease, heart disease, and the like.

According to some embodiments, the method may include communicating with a database having data associated with the mental health of subjects that have received one or more psychotherapeutic treatment. According to some embodiments, the database may include one or more scores, such as one or more post-initiation scores and/or enrollment scores of subjects that have received one or more psychotherapeutic treatment. According to some embodiments, the database may include one of more scores of the subjects that have received one or more psychotherapeutic treatment, wherein the scores are associated with the mental health of subjects at different times after initiation of the treatments, such as, for example, at one or more times ranging from 2 to 16 weeks after initiation of the treatment. According to some embodiments, the database may include clinical, demographic, and/or behavioral data associated with the subjects that have received one or more psychotherapeutic treatment. According to some embodiments, the database may include clinical, demographic, and/or behavioral data associated with the subjects at different times after initiation of the treatments, such as, for example, at one or more times ranging from 2 to 16 weeks after initiation of the treatment.

According to some embodiments, the method may include selecting a subset of comparable subjects from the data and/or scores associated with the subjects in the database. According to some embodiments, the method may include selecting the similarities relevant for the treated subject and/or specified therapeutic treatment given to the treated subject. For example, for a treated subject that is being treated with Citalopram, the method may include selecting comparable subjects which include subjects that have been treated with Citalopram. For example, for a treated subject that has been diagnosed with diabetes, the method may include selecting comparable subjects which were also diagnosed with diabetes. According to some embodiments, the similarities may include a similar and/or comparable medical background to the treated subject. According to some embodiments, the similarities may include having any one or more of a similar and/or comparable level of severity, symptoms, duration of symptoms prior to initiation of treatment, age, medical history, sex, and behavioral characteristics. According to some embodiments, the group of comparable subjects may include random subjects from the database. According to some embodiments, the group of comparable subjects may include all or most of subjects in the database.

According to some embodiments, the method may include obtaining one or more control scores associated with the mental health of the comparable subjects. According to some embodiments, the method may include obtaining a control score associated with the mental health of comparable subjects that have a comparable medical background to the treated subject. According to some embodiments, the comparable subjects may include subjects having any one or more of a similar and/or comparable level of severity, symptoms, duration of symptoms prior to initiation of treatment, age, medical history, sex, and behavioral characteristics. According to some embodiments, the method may include extracting the one or more control scores associated with the mental health of comparable subjects from a database. According to some embodiments, the comparable subjects may include subjects that had a previous response to other psychotherapeutic treatment(s), wherein the previous response of the comparable subjects were similar to and/or comparable with a previous response of the treated subject, wherein the previous response of the treated subject and the comparable subjects were in response to a same and/or comparable psychotherapeutic treatment(s).

For example, for a treated subject that was unresponsive to Citalopram and is now beginning treatment with sertraline can be compared to comparable subjects which were unresponsive to Citalopram and were then treated with sertraline.

According to some embodiments, the database may be associated with a memory module. According to some embodiments, the database may include one or more control scores associated with one or more specified psychotherapeutic treatments. According to some embodiments, the one or more control scores may be based, at least in part, on data associated with a plurality of subjects.

According to some embodiments, the one or more post-initiation scores, the initial treatment score, the enrollment score, and/or the score associated with the mental health of comparable subjects that have received the specified psychotherapeutic treatment, are based on different types of data. According to some embodiments, and as described in greater detail elsewhere herein, the post-initiation score and the one or more control scores may have the same units and/or be comparable. According to some embodiments, the post-initiation score and the one or more control scores may be comparable regardless of the type of data on which the scores are obtained. According to some embodiments, the post-initiation score and the one or more control scores may be based on different types of data. For example, the one or more control scores may be based on data inputted by a plurality of users (such as a questionnaire) and the post-initiation score may be obtained passively from an electronic device of the treated subject, each of the one or more control scores and the post-initiation score may include a rank between 1 and 40, a vector which may include the same relational axes, and the like.

According to some embodiments, the database may include one or more control scores associated with one or more medical backgrounds and/or pathologies, diagnosis, and/or previous treatments. According to some embodiments, the database may include control scores of a plurality of subjects prior to treatment, post-initiation of the treatment, and/or between 1 day to 16 weeks post initiation of the treatment. According to some embodiments, the one or more control scores within the database may include labels associated with any one or more of a time stamp indicating when the score was obtained in relation to initiation of the treatment, the type of treatment, previous treatment(s), initial level of severity of the subject, and/or the obtained data of the subject, such as the subjective data, the objective data, the behavioral data, the demographic data, the clinical data, or any combination thereof. According to some embodiments, the one or more control scores may include labels associated with a pre-known ratio of responsive to non-responsive subjects to the specified psychotherapeutic treatment. For example, a psychotherapeutic treatment may have a ratio of responsive to non-responsive subjects of 1:1, 2:3, or 4:5. According to some embodiments, the ratio of responsive to non-responsive subjects may be associated with data of clinical trials, clinical research, medical histories of one or more subjects that had been treated with the specified psychotherapeutic treatment, and the like.

According to some embodiments, the method may include generating a model based, at least in part, on the control scores. According to some embodiments, the method may include generating a model based, at least in part, on the control scores of the selected comparable subjects. According to some embodiments, the model may include one or more representations of a plurality of control scores of comparable subjects. According to some embodiments, and as described in greater detail elsewhere herein, the one or more representations may include one or more mathematical manipulations of the plurality of control scores of comparable subjects.

According to some embodiments, the model may include a non-linear model based, at least in part, on one or more of the post-initiation scores, the initial treatment score and/or the enrollment scores associated with the mental health of the comparable subjects. According to some embodiments, the non-linear model may include an exponential model. According to some embodiments, the non-linear model may include a graph, such as a graph having two or three dimensions. According to some embodiments, the model may include applying a curve fitting algorithm.

According to some embodiments, the method may include generating and/or obtaining an individual model for each of the comparable subjects. According to some embodiments, method may include generating and/or obtaining a cumulative model based on one or more individual models associated with the comparable subjects. According to some embodiments, the cumulative model may be based, at least in part, on the pre-known ratio of responsive to non-responsive subjects to the specified psychotherapeutic treatment.

According to some embodiments, the model may be pre-generated and stored in the database and/or a memory module. According to some embodiments, the method may include obtaining the model from the database and/or the memory module. According to some embodiments, the method may include comparing the one or more scores of the treated subject (i.e., one or more of the post-initiation scores and/or the enrollment score) to the model. According to some embodiments, the model may be configured to enable classifying the one or more scores of the treated subject, wherein the classifications may include a likelihood of the subject to respond to a psychotherapeutic treatment.

According to some embodiments, the method may include comparing the obtained score of the treated subject to the one or more control scores while taking into account the ratio of responsive to non-responsive subjects to the specified psychotherapeutic treatment. According to some embodiments, the method may include dividing the one or more control scores according to the ratio of responsive to non-responsive subjects to the specified psychotherapeutic treatment. For example, according to some embodiments, for a ratio of 50:50 in responsive to non-responsive subjects to the specified psychotherapeutic treatment, the control scores may be divided to two halves, each corresponding with responsive and non-responsive subjects, respectively.

According to some embodiments, the method may include comparing the one or more post initiation scores and/or the enrollment score of the treated subject with the control scores that have been divided in accordance with the ratio of responsive to non-responsive subjects to the specified psychotherapeutic treatment.

According to some embodiments, the method may include generating a model associated with the mental health of the treated subject based, at least in part, on at least one of the post initiation score, the second post initiation score, the enrollment score, or any combination thereof, of the treated subject. According to some embodiments, the model may be generated based on only one of the post initiation score, the second post initiation score, and the enrollment score. According to some embodiments, the model may include a non-linear model based, at least in part, on one or more of the post-initiation scores, the initial treatment score and/or the enrollment scores associated with the mental health of the treated subject. According to some embodiments, the non-linear model may include an exponential model. According to some embodiments, the non-linear model may include a graph, such as a graph having two or three dimensions. According to some embodiments, the model may include applying a curve fitting algorithm.

According to some embodiments, the method may include comparing the model associated with the mental health of the treated subject with the cumulative model and/or a plurality of individual models of the comparable subjects. According to some embodiments, the method may include comparing one or more parameters of the model selected from the group of: slope, area under curve, plateau, maximal slope, minimal slope, average slope, median slope, standard deviation, correlation coefficient and variance of the model associated with the mental health of the treated subject with a corresponding parameter of the cumulative model and/or a plurality of individual models of the comparable subjects.

According to some embodiments, at step 106, the method may include classifying the subject as responsive or non-responsive to the specified psychotherapeutic treatment. According to some embodiments, the method may include classifying the subject as responsive or non-responsive to the specified psychotherapeutic treatment based, at least in part, on the comparison of the post-initiation score to a score associated with the mental health of comparable subjects that have received the specified psychotherapeutic treatment, such as the one or more control scores. According to some embodiments, the method may include classifying the subject as responsive or non-responsive to the specified psychotherapeutic treatment based, at least in part, on the comparison between the model associated with the mental health of the treated subject and the cumulative model and/or a plurality of individual models of the comparable subjects.

According to some embodiments, the method may include applying the one or more scores of the treated subject to a classification algorithm. According to some embodiments, the classification algorithm may be configured to generate the model associated with the mental heal of the treated subject. According to some embodiments, the classification algorithm may be configured to compare the one or more scores of the treated subject to the cumulative model and/or compare the generated model associated with the treated subject to the cumulative model.

According to some embodiments, the classification algorithm may be configured to receive the obtained data associated with the one or more scores. According to some embodiments, the classification algorithm may be configured to select the similarities of the group of comparable subjects as described above. According to some embodiments, the classification algorithm may be configured to select the similarities based, at least in part, on the obtained data and/or the data in the database.

According to some embodiments, the classification algorithm may be configured to receive the one or more scores and/or model of the treated subject and output a detection associated with a treatment outcome of the treated subject. According to some embodiments, the classification may be based on the pre-known ratio of responsive to non-responsive subjects to the specified psychotherapeutic treatment.

According to some embodiments, the classification algorithm may be configured to receive any one or more of the enrollment score, post initiation score, and/or initiation score of the treated subject. According to some embodiments, the classification algorithm may be configured to receive one or more scores obtained from the treated subject at 0 to 4 weeks post-initiation of the psychotherapeutic treatment. According to some embodiments, the classification algorithm may be configured to output a detected response for the treated subject. According to some embodiments, the detected response may include a detection of the response of the treated subject to the psychotherapeutic treatment at one or more of 1, 2, 3, 4, 5, 6, 7, 8, 9, 10, 11, 12, 13, 14, 15, 16, and 17 weeks post treatment. According to some embodiments, the sensitivity of the detection may range between 50-97%.

According to some embodiments, the method may include taking into account the initial level of severity of the subject prior to receiving the psychotherapeutic treatment. According to some embodiments, the method may include taking into account the initial level of severity of the subject prior to receiving the psychotherapeutic treatment in relation to the initial level of severity associated with the one or more control score associated with comparable subjects that have received the specified psychotherapeutic treatment. According to some embodiments, the method may include comparing the initial level of severity of the subject with the initial level of severity associated with the one or more control score associated with comparable subjects that have received the specified psychotherapeutic treatment. According to some embodiments, the method may include comparing the initial level of severity of the subject with the initial level of severity associated with the one or more scores on which the control score may be based.

According to some embodiments, the method may include deriving a parameter from the model associated with the treated subject. According to some embodiments, the parameter may include any one or more of the slope, area under curve, plateau, maximal slope, minimal slope, average slope, median slope, standard deviation, correlation coefficient and variance of the model. According to some embodiments, the parameter may be associated with a degree of change in a behavior of the treated subject. According to some embodiments, the method may include classifying the subject as responsive or non-responsive to the specified psychotherapeutic treatment based, at least in part, on a comparison of the parameter to a parameter associated with the mental health of comparable subjects that have received the specified psychotherapeutic treatment. According to some embodiments, the method may include deriving the parameter associated with the mental health of comparable subjects that have received the specified psychotherapeutic treatment from the cumulative model and/or the plurality of individual models.

According to some embodiments, the method may include providing a weight to the parameter and/or to the post-initiation score and/or the initial treatment score and/or the enrollment score based on a general change in behavior of a population of individuals to which the subject belongs, the population of individuals comprising individuals who are not diagnosed with a mental disease. For example, if it is known that unemployed subjects undergoing psychotherapeutic treatment respond about 30% less to a treatment (or in other words, being unemployed makes a subject more likely to be a non-responder to a treatment when compared with a comparable subject who is employed), then for the unemployed treated subject, the parameter and/or to the post-initiation score and/or the initial treatment score and/or the enrollment score may be multiplied by a value such that the detection of the response of responsiveness to a treatment may correspond with the reduction in responsiveness that is associated with being unemployed.

According to some embodiments, the method may include predicting a response of the treated subject to a second psychotherapeutic treatment in the case that that the subject is classified as non-responsive to a first psychotherapeutic treatment. According to some embodiments, the method may include predicting a second psychotherapeutic treatment for which the treated subject will be classified as responsive. According to some embodiments, the method may include predicting a second psychotherapeutic treatment for which the treated subject will have a higher responsiveness level. For example, a subject that is being treated with Citalopram may be classified as unresponsive within two or four weeks of initiating the Citalopram treatment. The classification may then recommend starting a different treatment using Sertraline based on other comparable subjects that may have been unresponsive to Citalopram and were then responsive to Sertraline.

According to some embodiments, the program code of the system may include a classifying algorithm configured to classify the treated subject as responsive or non-responsive to the psychotherapeutic treatment. According to some embodiments, the algorithm may be configured to receive data associated with the mental health of the treated subject. According to some embodiments, the algorithm may be configured to receive inputted data from the treated subject. According to some embodiments, the algorithm may be configured to collect data from an electronic device of the treated subject, such as a smartphone of the treated subject.

According to some embodiments, the algorithm may be configured to calculate any one or more of the post-initiation score, the second post-initiation score, and the enrollment score of the treated subject based, at least in part, on the received and/or collected data. According to some embodiments, the algorithm may be configured to generate the model using one or more of the post-initiation score, the second post-initiation score, and the enrollment score of the treated subject. According to some embodiments, the algorithm may be configured to access the database and/or the memory module. According to some embodiments, the algorithm may be configured to identify and/or select comparable subjects from the database and/or memory module. According to some embodiments, the algorithm may be configured to calculate one or more of the post-initiation scores, the second post-initiation scores, and the enrollment scores of the comparable subjects (or control scores). According to some embodiments, the algorithm may be configured to generate one or more model based on the control scores. According to some embodiments, the algorithm may be configured to classify the treated subject as responsive or non-responsive to the psychotherapeutic treatment based on a comparison of the one or more scores of the treated subject with the one or more control scores. According to some embodiments, the algorithm may be configured to classify the treated subject as responsive or non-responsive to the psychotherapeutic treatment based on a comparison of the generated model of the treated subject to the generated model of the comparable subjects.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications, and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

### EXAMPLES

### Example 1

### Mathematical Modelling of Response (Exponential) VS. Classic Response as Detectors

In the examples, the term “model” or “exponential model” refer to the comparison of the scores of the treated subjects to the scores of comparable subjects as described hereinabove.

The term “classic response detection” refers to a commonly used technique for detection of responsiveness of a subject to a treatment, in which subjects are scored based on a questionnaire, and a “classic responder” refers to an improvement of 50% or more in the subject's depression score.

Table 1 compares between the results of the classic response detection and the model as described in the present application, using exponential modeling.

The calculations as depicted below are based on an average of 8 different treatments analyses on STAR*D data.

The classic response of week 2 includes scores of the treated subjects during (and/or up to) week 2 of the treatment, and were used to classify (or detect) the subject's responsiveness to the treatment in weeks 6, 9, and 12. The classic response of (and/or up to) week 4 includes scores of the treated subjects during week 4 of the treatment, and were used to classify (or detect) the subject's responsiveness to the treatment in weeks 6, 9, and 12. These results were then compared to the classical technique, to see if the subjects in weeks 2 and 4 would have been classified as responders in weeks 6, 9, and 12 using the classic response detection.

The mathematical modelling of response (i.e., “exponential modelling”) could detect already by week 2 the “classic responders” of week 6 (i.e., patients that experienced a drop of >=50% of their depression severity score in week 6) with an average balanced accuracy of 65.5%, comprised of sensitivity of 74.3% and specificity of 56.6%.

In contrast, the “classic response” definition in week 2 was able to detect classic responders of week 6 with an average balanced accuracy of 60%, sensitivity of only 27.3% and specificity of 92.8%. The extremely low sensitivity of the classic response detection abilities means that most classic responders in week 6 could not have been detected as responders using the same classic definition of response at the beginning of treatment. However, the same subjects that would have been responders in week 6 and were not classified as responders using the classic response detection would have been classified as responsive to the treatments in week 2 using the exponential modeling.

The balanced accuracy of the exponential modelling detecting for later (future) weeks appears to be more stable, decreasing in “slower pace” than the classic response detecting later weeks.

As seen in Table 1, the gap (or difference) between the two approaches in the earlier week(s) becomes larger as the week number is increased, or in other words, the classic response detection becomes less accurate in comparison with the exponential model when the period between the week in which the scores are obtained and the week for which the detection is made increases.

The sensitivity (i.e., the accuracy of detecting the responders of the later week by the response definition of the earlier week) is much higher using the exponential definition/modelling of the earlier week. As seen in table 1, almost all patients in the earlier weeks are considered as non-responders by the classic response detection. In weeks 2 and 4, the exponential model can identify subjects that will become responders later in the treatment (such as weeks 6, 9, and 12) which the classic response detection cannot identify these subjects.

TABLE 1
The results of the classic response detection and the model as described
in the present application, using exponential modeling

| Detection | | Balanced | | |
|---|---|---|---|---|
| week | Type of response detection | accuracy | Sensitivity | Specificity |
| Detection | Exponential modelling of week 2, | 65.5% | 74.3% | 56.6% |
| from | detecting classic response of week 6 | | | |
| week | Classic response of week 2, detecting | 60% | 27.3% | 92.8% |
| 2 data | classic response of week 6 | | | |
| | Exponential modelling of week 2, | 65.8% | 72% | 59.7% |
| | detecting classic response of week 9 | | | |
| | Classic response of week 2, detecting | 60.1% | 25.5% | 94.7% |
| | classic response of week 9 | | | |
| | Exponential modelling of week 2, | 64.7% | 69.2% | 60.2% |
| | detecting classic response of week 12 | | | |
| | Classic response of week 2, detecting | 55.1% | 18.4% | 91.9% |
| | classic response of week 12 | | | |
| Detection | Exponential modelling of week 4, | 72.4% | 82.1% | 62.6% |
| from | detecting classic response of week 6 | | | |
| week | Classic response of week 4, detecting | 70.6% | 52.5% | 88.9% |
| 4 data | classic response of week 6 | | | |
| | Exponential modelling of week 4, | 69.7% | 76.2% | 63.2% |
| | detecting classic response of week 9 | | | |
| | Classic response of week 4, detecting | 65.6% | 42.1% | 89.1% |
| | classic response of week 9 | | | |
| | Exponential modelling of week 4, | 64.4% | 68.9% | 59.8% |
| | detecting classic response of week 12 | | | |
| | Classic response of week 4, detecting | 57.8% | 31.9% | 83.7% |
| | classic response of week 12 | | | |

The specificity (i.e., the accuracy of detecting the non-responders of the later weeks by the response definition of the earlier week) is lower using the exponential definition/modelling of the earlier week, since most non-responders of later weeks were also non-responsive according to the classic response in earlier weeks, thereby showing that the specificity is so high in the classic response definition.

The specificity of the classic response detection appears higher than the specificity of the exponential model due to the high rates of classification of non-responders using the classic response detection. The classic response detection classifies non-responders at a higher rate than responders, thereby “achieving” a higher s proportion of subjects that are non-responsive to a treatment and are classified as non-responders to the treatment.

The sensitivity of the exponential model is higher than that of the classic response detection by about 50% for detections made in week 2 after initiation of the treatments and by about 30%-35% for detections made in week 4 after initiation of the treatments, meaning that the exponential model has a higher proportion of subjects that are in fact responsive to the treatment and were classified as responsive to the treatment in its earlier weeks.

### Example 2

### Mathematical Modelling of Response (Exponential) VS. Classic Response as Detectors

In this experiment, the exponential model was calculated and the treated subjects were classified as “future” responders and non-responders, such as described above.

Table 2 shows the positive predictive values (PPV) and negative predictive values (NPV) for discrepancy subjects, which are subjects that were detected to be non-responders by the classic response detection but detected to be responders by the exponential modeling.

The calculations as depicted below are based on an average of 8 different treatments analyses on STAR*D data, wherein the average across treatments includes a same weight for each treatment when the average was calculated, and the cumulative average includes a weighted average in which treatments were weighted with respect to the number of treated subjects of the specific treatment (such that treatments with more subjects in its test group weighted more into the average than treatments with less subjects in its test group).

TABLE 2
the positive predictive values (PPV) and negative predictive values (NPV) for the discrepancy subjects

| | Type of response | | | | | | |
|---|---|---|---|---|---|---|---|
| | detection (for the | | | | | | |
| | discrepancy patients: | Average | PPV of the | NPV of the | | | |
| | detected to be non- | QIDS | exponential | classic | | | |
| | responders by the | reduction | modelling | response | Average | PPV of the | NPV of the |
| | classic response, and | (average | (average | (average | QIDS | exponential | classic |
| Detection | responders by the | across | across | across | reduction | modelling | response |
| week | exponential modelling) | treatments) | treatments) | treatments) | (cumulative) | (cumulative) | (cumulative) |
| Detection | Detecting classic | 34.7% | 34.9% | 65.1% | 45.2% | 48% | 52% |
| from | response of week 6 | | | | | | |
| week 2 | Detecting classic | 39.2% | 41.5% | 58.5% | 50.9% | 57.6% | 42.4% |
| data | response of week 9 | | | | | | |
| | Detecting classic | 47.6% | 58.8% | 41.2% | 56.3% | 68.4% | 31.6% |
| | response of week 12 | | | | | | |
| Detection | Detecting classic | 38.6% | 35.5% | 64.5% | 44% | 44.7% | 55.3% |
| from | response of week 6 | | | | | | |
| week 4 | Detecting classic | 39.6% | 46.7% | 53.3% | 48.4% | 55.9% | 44.1% |
| data | response of week 9 | | | | | | |
| | Detecting classic | 51.3% | 60.2% | 39.8% | 56.8% | 67.1% | 32.9% |
| | response of week 12 | | | | | | |

The significance of this experiment is in that the vast majority of the patients are detected to be non-responders according to the classic response in the beginning of treatment, in contrast to the exponential modeling that is able to detect about 50% of all patients to be responders.

It is shown in table 2 that in both of the average across different treatments and in a cumulative average, that many of the treated subjects are responders by week 12 (˜60% in the average-across-treatments analysis, and ˜67%-68% in the cumulative analysis)—i.e., these are patients that would have been mostly “missed” if the detection was to be made using the classic response as the main detector for them in earlier weeks.

The exponential model therefore enables a more accurate detection for which subjects will become responders by week 12 of the treatment in relation to the detections of the classic response detection.

Table 3 shows the positive predictive values (PPV) and negative predictive values (NPV) for discrepancy subjects receiving either Citalopram and/or Sertraline, and Table 4 shows the positive predictive values (PPV) and negative predictive values (NPV) for subjects receiving Citalopram.

TABLE 3
positive predictive values (PPV) and negative predictive values (NPV)
for discrepancy subjects receiving either Citalopram or Sertraline

| | Type of response detection | Average | PPV of the | NPV of the |
|---|---|---|---|---|
| | (for the discrepancy patients: | QIDS | exponential | classic |
| | detected to be non- | reduction | modelling | response |
| | responders by the classic | (average | (average | (average |
| Detection | response, and responders by | of the two | of the two | of the two |
| week | the exponential modelling) | treatments) | treatments) | treatments) |
| Detection | Detecting classic response | 42.5% | 43% | 57% |
| from | of week 6 | | | |
| week 2 | Detecting classic response | 46.2% | 50.9% | 49.1% |
| data | of week 9 | | | |
| | Detecting classic response | 56.9% | 69.1% | 30.9% |
| | of week 12 | | | |
| Detection | Detecting classic response | 40.8% | 34.9% | 65.1% |
| from | of week 6 | | | |
| week 4 | Detecting classic response | 45.1% | 51.7% | 48.3% |
| data | of week 9 | | | |
| | Detecting classic response | 55.7% | 63.4% | 36.6% |
| | of week 12 | | | |

As seen in Table 3, the positive predictive values (PPV) for discrepancy subjects receiving either Citalopram or Sertraline reaches 69.1% for a detection for week 12 that is made in week 2 and 63.4% for a detection for week 12 that is made in week 4, meaning that the exponential model used identifies 69.1% and 63.4% responsive subjects that would have been classified as non-responsive in the classic response detection. The prediction values for Citalopram and Sertraline combined showed overall higher PPV of the exponential modelling in relation to the PPV of the exponential modeling of all 8 treatments as seen in Table 2. This may be due to the higher number of subjects in the experiment group which belonged to the treatment groups associated with Citalopram and Sertraline, thereby indicating that the exponential model may have an even higher accuracy and sensitivity for higher databases of treated subjects.

TABLE 4
positive predictive values (PPV) and negative predictive values (NPV) for subjects receiving Citalopram

| | Type of response | | | | | | % of |
|---|---|---|---|---|---|---|---|
| | detection | | | | | | response |
| | (for the discrepancy | | | | | Average | in the |
| | patients: detected to be | | | | | QIDS | relevant |
| | non-responders by the | | | NPV of | | reduction | week (out |
| | classic response, and | Average | PPV of the | the | | (for all | of all |
| Detection | responders by the | QIDS | exponential | classic | General | citalopram | citalopram |
| week | exponential modelling) | reduction | modelling | response | statistics | patients) | patients) |
| Detection | Detecting classic response | 50.6% | 55% | 45% | Week 6 | 43% | 45.5% |
| from | of week 6 | | | | | | |
| week 2 | Detecting classic response | 56.5% | 66.1% | 33.9% | Week 9 | 49.3% | 55.8% |
| data | of week 9 | | | | | | |
| | Detecting classic response | 61.2% | 74% | 26% | Week 12 | 56.7% | 66.8% |
| | of week 12 | | | | | | |
| Detection | Detecting classic response | 48.6% | 52.6% | 47.4% | Week 6 | 43% | 45.5% |
| from | of week 6 | | | | | | |
| week 4 | Detecting classic response | 55.7% | 66.4% | 33.6% | Week 9 | 49.3% | 55.8% |
| data | of week 9 | | | | | | |
| | Detecting classic response | 61.7% | 74.4% | 25.6% | Week 12 | 56.7% | 66.8% |
| | of week 12 | | | | | | |

As seen in Table 4, the positive predictive values (PPV) for subjects receiving Citalopram reaches 74% and 74.4% for detection for week 12 that are made in weeks 2 and 4, respectively, using the exponential model. In contrast, the negative predictive values (NPV) for subjects receiving Citalopram are 26% and 25.6% for detections for week 12 that are made in weeks 2 and 4, respectively, using the classic response detection.

The prediction values for Citalopram combined showed overall higher PPV of the exponential modelling in relation to the PPV of the exponential modeling of all 8 treatments as seen in Table 2 and of the PPV of the exponential modeling of the Citalopram and Sertraline treatments in Table 3. This may be due to the higher number of subjects in the experiment group which belonged to the treatment group associated with Citalopram, thereby indicating that the exponential model may have an even higher accuracy and sensitivity for higher databases of treated subjects.

### Example 3

### Mathematical Modelling of Response (Exponential) VS. Classic Response as Detectors

In this experiment, the exponential model was calculated and the treated subjects were classified as responders and non-responders, such as described above.

The subjects in this experiment have received a first treatment (level 1), were classified as unresponsive to the first treatment in level 1, and were then given a second treatment (level 2). Table 5 shows the accuracy, sensitivity, and specificity of the detection for the level 2 treatment of the subjects using both the exponential model and the classic response detection. The last column of Table 5 shows the specificity of the detection for the same subject (which was non-responsive) in the level 1 treatment (i.e., to the Citalopram treatment)

| | Type of response Detection | Balanced | | | |
|---|---|---|---|---|---|
| Detection | (for level 2, only patients | accuracy | Sensitivity | Specificity | Specificity |
| week | who failed level 1) | (level 2) | (level 2) | (level 2) | (level 1) |
| Detection | Exponential modelling of week 2, | 63.3% | 71% | 55.6% | 68.5% |
| from | detecting classic response of week 6 | | | | |
| week 2 | Classic response of week 2, detecting | 58.4% | 23.2% | 93.5% | 94.1% |
| data | classic response of week 6 | | | | |
| | Exponential modelling of week 2, | 66.7% | 73.7% | 59.6% | 66.6% |
| | detecting classic response of week 9 | | | | |
| | Classic response of week 2, detecting | 59.9% | 24.8% | 95.1% | 94.1% |
| | classic response of week 9 | | | | |
| | Exponential modelling of week 2, | 64.4% | 69.7% | 59% | 63.2% |
| | detecting classic response of week 12 | | | | |
| | Classic response of week 2, detecting | 54.3% | 17.5% | 91.2% | 92.8% |
| | classic response of week 12 | | | | |
| Detection | Exponential modelling of week 4, | 71.7% | 81.9% | 61.5% | 82.1% |
| from | detecting classic response of week 6 | | | | |

The balanced accuracy, sensitivity and specificity are much more stable for the exponential modelling than the classic response detection later weeks (such as weeks 9 and 12). Additionally, the gap between the two detection approaches in the earlier week becomes larger as the detection week number increases. The sensitivity (i.e., the accuracy of detecting the responders of the later week by the response definition of the earlier week) is much higher using the exponential definition/modelling of the earlier week.

Table 5 shows that the specificity for the subject from level 1 ranges between 68.5% to 82.1%, and the sensitivity for these subjects in level 2 than ranges from 68.8% to 81.9%, indicating that exponential model was able to detect that these subjects would be unresponsive in level 1 of treatment and then will be responsive in the level 2 of treatment in the early weeks of each treatment.

1.-24. (canceled)

25. A method for early detection of a psychotherapeutic treatment response for a subject in need thereof, the method comprising:
obtaining data associated with a level of severity of a mental health of the subject four weeks or less after initiation of the psychotherapeutic treatment;
calculating a post-initiation score associated with the subject's mental health based on the obtained data; and
classifying the subject as responsive or non-responsive to the specified psychotherapeutic treatment based, at least in part, on a comparison of the post-initiation score to a score associated with the mental health of comparable subjects that have received the specified psychotherapeutic treatment.

26. The method according to claim 25, wherein the predetermined time includes two weeks or less after initiation of psychotherapeutic treatment.

27. The method according to claim 25, wherein the obtained data comprises subjective and/or objective data associated with the level of severity of a mental health of the subject.

28. The method according to claim 27, wherein the subjective obtained data comprises data inputted by the subject and the objective obtained data comprises passive/behavioral data collected from the subject, wherein the passive data comprises sleep characteristics, level of activity, and smartphone usage.

29. The method according to claim 27, wherein the data further comprises clinical and demographic data of the subject.

30. The method according to claim 25, wherein classifying is further based on a pre-known ratio of responsive to non-responsive subjects to the specified psychotherapeutic treatment.

31. The method according to claim 25, wherein classifying further comprises taking into account the initial level of severity of the subject in relation to the initial level of severity associated with the plurality of scores associated with comparable subjects that have received the specified psychotherapeutic treatment.

32. The method according to claim 25, wherein the comparable subjects comprise subjects having any one or more of a similar and/or comparable level of severity, comparable symptoms, comparable duration of symptoms prior to initiation of treatment, comparable age, comparable medical history, same sex, and comparable behavioral characteristics.

33. The method according to claim 25, wherein the post-initiation score and the score associated with the mental health of comparable subjects that have received the specified psychotherapeutic treatment, are based on different types of data.

34. The method according to claim 25, further comprising obtaining data associated with a level of severity of a mental health of the subject at another time within four weeks or less of initiation of the specified psychotherapeutic treatment, and calculating an initial treatment score associated with the subject's mental health based on the obtained data.

35. The method according to claim 25, further comprising obtaining data associated with a level of severity of a mental health of the subject prior to the initiation of the specified psychotherapeutic treatment, and calculating an enrollment score associated with the subject's mental health based on the obtained data.

36. The method according to claim 35, wherein the post-initiation score and the initial treatment score and/or the enrollment score are based on different types of data.

37. The method according to claim 35, wherein the post-initiation score, the initial treatment score, the enrollment score, and/or the score associated with the mental health of comparable subjects that have received the specified psychotherapeutic treatment, are based on different types of data.

38. The method according to claim 37, further comprising generating a non-linear model based, at least in part, on the post-initiation score and the initial treatment score and/or the enrollment score.

39. The method according to claim 38, wherein the non-linear model is an exponential model.

40. The method according to claim 38, further comprising deriving a parameter from the non-linear model, wherein the parameter comprises at least one of a slope, area under curve, plateau, maximal slope, minimal slope, average slope, median slope, standard deviation, correlation coefficient and variance.

41. The method according to claim 40, wherein the parameter is associated with a degree of change in a behavior of the subject.

42. The method according to claim 38, further comprising providing a weight to the parameter and/or to the post-initiation score and/or the initial treatment score and/or the enrollment score based on a general change in behavior of a population of individuals to which the subject belongs, the population of individuals comprising individuals who are not diagnosed with a mental disease.

43. The method according to claim 38, further comprising classifying the subject as responsive or non-responsive to the specified psychotherapeutic treatment based, at least in part, on a comparison of the parameter to a parameter associated with the mental health of comparable subjects that have received the specified psychotherapeutic treatment.

44. The method according to claim 25, wherein the comparable subjects comprise subjects that had a previous response to other psychotherapeutic treatment(s) comparable to a previous response of the subject to a similar and/or comparable psychotherapeutic treatment(s), prior to receiving the specified psychotherapeutic treatment and/or a psychotherapeutic treatment comparable with the psychotherapeutic treatment.
